(19) United States
(12) Patent Application Publication
(10) Pub. No.: US 2006/0018962 A1
(43) Pub. Date: Jan. 26, 2006

(54) Sustained release formulation of tramadol

(76) Inventors: Reza Eivaskhani, Freiburg i.Br (DE); Christian Braun, Rielasingen-Worbingen (DE); Stefan Merkle, Gottmadingen (DE)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 10/508,615

(22) PCT Filed: Mar. 21, 2003
(86) PCT No.: PCT/EP03/03050

§ 371 Date: Aug. 11, 2005

(30) Foreign Application Priority Data
Mar. 22, 2002 (EP) 02076130.0

(51) Int. Cl. A61K 31/137; A61K 9/22

(52) U.S. Cl. 424/468; 514/650

(57) ABSTRACT

This invention relates to sustained release oral dosage forms comprising tramadol or a salt thereof dispersed in a matrix, wherein said matrix comprises xanthan gum.

### BRIEF DESCRIPTION OF THE INVENTION

This invention relates to sustained release oral dosage forms comprising tramadol or a salt thereof dispersed in a matrix, wherein said matrix comprises xanthan gum.

### BACKGROUND OF THE INVENTION

Controlled release formulations have been introduced for active ingredients that require a constant release without peaks or drops in the release of the active ingredient during a certain period of time. A variety of controlled release formulations are now available that avoid temporary over- or under dosing of the active ingredient.

So-called sustained release formulations have been developed in which the release of the active substance is prolonged in some way in order to maintain therapeutic activity for a longer period of time. Sustained release formulations typically are applied for drugs that have a short half-life or for actives that require active blood plasma levels for long periods of time. In this way, multiple daily dose regimens can be avoided such as b.i.d. and q.i.d regimens, which often lead to problems caused by lack of patient compliance. The term ‘sustained release’ is also often used for formulations that show controlled release during a prolonged period of time.

A class of analgesic cycloalkanol-substituted phenol esters having a basic amine group in the cycloalkyl ring, are disclosed in U.S. Pat. No. 3,652,589. Among these is the compound (1R,2Ror 1S,2S)-2-[(dimethylamino)methyl]-1-(3-methoxyphenyl) cyclohexanol, commonly known as tramadol, which is specifically disclosed therein.

A series of articles pertaining to the pharmacology, toxicology and clinical studies of tramadol are found in Arzneim. Forsch., (Drug Res.), 1978, 28(1), 114. Tramadol hydrochloride has been reported to be an orally active pure agonist opioid analgesic. However, clinical experience indicates that tramadol lacks many of the typical side effects of opioid agonists, e.g., respiratory depression, constipation, tolerance and abuse liability. Tramadol's ‘atypical’ combination of non-opioid and opioid activity makes tramadol a very unique drug. Tramadol is currently marketed as an analgesic.

One of the problems associated with tramadol is that it has a relatively short half-life thus requiring a multiple dose regimen. Initial overdosing during the initial time period after administration may result in side effects whereas under dosing results in inefficacy so that the pain sensation may arise again. Hence there is a need for sustained release formulations that release tramadol over longer periods of time.

U.S. Pat. No. 5,601,842 discloses matrix formulations of tramadol or a tramadol salt. Sustained release formulations of tramadol in wax-like materials have been described in U.S. Pat. No. 6,306,438. EP-A-699,436 discloses a number of controlled release formulations of tramadol. JP 08/295637 reported in Derwent Publications Section Ch, Week 199704, Class AN 96, AN 1997-037974, discloses topical formulations for application in the mouth that may comprise a series of analgesics, i.a. tramadol hydrochloride and a series of macromolecules, i.a. xanthan gum. U.S. Pat. No. 6,340,475 in turn discloses oral dosage forms in which drugs are incorporated in matrixes comprised of hydrophilic polymers that swell upon imbibition of water. A number of actives are mentioned for incorporation in this system, one of which is tramadol.

However, there is a need for further sustained release formulations of tramadol that allow the controlled release of tramadol active during specified longer periods of time and preferably in a reproducible manner. In particular there is a need for formulations that release tramadol during 12 hours and preferably during 24 hours. Further there is a need for sustained release formulations that release tramadol active in a controlled manner, i.e. without peaks or drops in its release pattern.

Providing sustained release formulations that fulfill these needs is a desirable goal to achieve, which is attained by the formulations of the present invention.

A further object of the present invention is to provide a method for treating conditions of pain, in particular severe pain, in mammals.

### SUMMARY OF THE INVENTION

This invention relates to a sustained release oral pharmaceutical dosage form containing an effective amount of tramadol, or a pharmaceutically acceptable salt thereof, dispersed in a matrix characterized in that the matrix comprises xanthan gum.

In a particular aspect, the invention relates to a sustained release oral pharmaceutical dosage form containing an effective amount of tramadol, or a pharmaceutically acceptable salt thereof, dispersed in a matrix wherein the carrier essentially consists of xanthan gum.

In a further aspect, the invention concerns a sustained release oral pharmaceutical dosage form containing an effective amount of tramadol, or a pharmaceutically acceptable salt thereof, dispersed in a matrix wherein the matrix contains from about 20% to about 90%, in particular from about 30% to about 80% of xanthan gum.

In a particular embodiment the dosage forms according to the present invention are coated with an appropriate taste masking coating.

The oral dosage forms of this invention are for administering to a human patient on a twice-a-day (b.i.d.) and preferably on a once-a-day basis.

In a preferred embodiment the oral pharmaceutical dosage form of the invention comprises defined unit doses, in particular tablets.

In another aspect the invention concerns a process for manufacturing an oral dosage form, as described herein, said process comprising mixing an effective amount of tramadol, or a salt form thereof, in solid form with xanthan gum and optional other ingredients, and converting the mixture into the desired form.

In a particular aspect the invention concerns a process for manufacturing an oral dosage form described herein, which is a tablet, said process comprising direct compression of a mixture of an effective amount of tramadol, or a salt form thereof, in solid form with xanthan gum and other ingredients. In case of direct compression the other ingredients preferably are a suitable flow enhancer and a suitable lubricant.

Furthermore, the invention concerns a method of treating a warm blooded animal suffering from analgesia, said method comprising the administration of an oral dosage form containing an effective amount of tramadol, said dosage form being as described herein.

### DETAILED DESCRIPTION OF THE INVENTION

Tramadol is the compound (1R,2R or 1S,2S)-2-[(dimethylamino)methyl]-1-(3-methoxyphenyl)-cyclohexanol. Preferably tramadol is used as a pharmaceutically acceptable salt form, in particular as its hydrochloride salt. Tramadol is commercially available from Gruenenthal or may be made by the process described in U. S. Pat. No. 3,652,589.

The dosage forms of the present invention may contain from about 10 mg to about 150 mg or from about 10 mg to 100 mg tramadol hydrochloride per unit, preferably from about 15 mg to about 75 mg of tramadol hydrochloride per unit, or from about 25 mg to about 80 mg or further in particular from about 25 mg to about 65 mg of tramadol hydrochloride per unit. In case of application of tramadol free base or other salts, an equivalent amount of active is used.

The dosage forms of the invention preferably contain specific amounts of xanthan gum. The quantity of xanthan gum in the dosage forms of the invention is selected in function of the quantity of tramadol in the dosage form and the desired release pattern. The dosage forms of the invention may contain quantities of xanthan gum which are in the range of about 50 mg to about 500 mg, in particular in the range of about 100 mg to about 300 mg, more in particular in the range of about 100 mg to about 200 mg.

It has been found that for a unitary dosage form containing about 90 mg of tramadol hydrochloride, a quantity of 160 mg of xanthan gum results in a release of 100% of the tramadol in 24 hours.

In a particular aspect, the oral dosage forms of the invention contain an effective amount of tramadol, or a pharmaceutically acceptable salt thereof, dispersed in a matrix wherein the matrix contains from about 20% to about 90%, in particular from about 30% to about 80% of xanthan gum. The percentages mentioned herein are w/w relative to the total weight of the dosage form.

The dosage forms of the invention in particular are orally applicable single unit dosage forms. Examples of such dosage forms are pills, capsules and tablets. Because of their ease in administration, tablets represent the most advantageous oral dosage unit form.

The dosage forms of the invention may additionally contain further ingredients such as starches, kaolin, lubricants, binders and the like. Preferred additional carriers are lubricants, e.g. magnesium stearate, flow enhancers or fillers, e.g. silica (silicon dioxide), fillers such as sugars, in particular lactose, titanium dioxide and the like.

Particular embodiments of this invention are dosage forms, in particular tablets, that contain as further ingredients lactose as a filler and magnesium stearate as a lubricant.

Lactose is added to improve compressibility of the blend. Magnesium stearate is added to avoid tablet sticking on the lower or upper punch during the compression.

The concentration of magnesium stearate in the dosage forms may vary but good results are obtained when adding this ingredient in amounts ranging from about 0.5 to about 1.0% (w/w relative to the total weight of the dosage form). The concentration of lactose in the dosage forms may vary but good results are obtained when adding it in amounts which range from about 5% to about 80%, preferably from about 10% to about 65%, more preferably from about 20% to about 50% (w/w relative to the total weight of the dosage form).

The dosage forms of the invention can be prepared by mixing tramadol or its salt form with xanthan gum while adding optional ingredients. The latter may also be added after the mixing of tramadol and xanthan gum. The thus obtained mixtures are subsequently worked into suitable dosage forms following art-known methods. In case of tablets, the mixture can be granulated and subsequently compressed.

An additional feature of the present invention comprises the finding that when using tramadol, or a salt thereof, and xanthan gum mixtures, the tablets can be prepared by direct compression. The mixtures for direct compression preferably contain a lubricant, in particular magnesium stearate. They may additionally contain a filler, in particular a sugar such as lactose. They may furthermore contain a flow enhancer such as colioidal silica (silicon dioxide). Apart from the required quantities of tramadol or a salt thereof, and xanthan gum, the mixtures for direct compression preferably also contain a flow enhancer and a lubricant, and optionally, a filler. In the mixtures for direct compression the lubricant preferably is present in concentrations in the range of about 0.75% to about 1.0%. The filler is present in concentrations from about 5% to about 80%, preferably from about 10% to about 65%, more preferably from about 20% to about 50%. The flow enhancer is present in concentrations from about 0.4% to about 0.6%, preferably about 0.45% to about 0.50%. All percentages herein are w/w relative to the total weight of the dosage form.

Of particular interest are mixtures comprising tramadol hydrochloride, xanthan gum, silicon dioxide, in particular colloidal silica, magnesium stereate and, optionally, lactose.

Preferred embodiments of the invention are tablets and more preferably these are coated, in particular film-coated. Coated tablets are easier to swallow than uncoated tablet cores, are usually easier to distinguish from other tablets—in particular when the film-coat contains a dye or a pigment—, and may furthermore have an improved stability (shelf-life). In the present instance coating is mainly is for taste masking purposes because of the bitter taste of tramadol. Coatings are applied using art known methods using art known materials usually applied for this purpose. Particularly attractive coating products are based on suitable film-forming polymers such as hydroxypropylmethylcellulose (HPMC) or polyvinylalcohol (PVA). Preferably, a plasticizer is added. Examples of suitable plasticizers are polyethylene glycol or derivatives thereof such as polyethoxylated alkylglycerides, e.g. polyethoxylated stearyl monoglyceride, in particular the material sold under the trade name Macrogol™. Further ingredients may be added to the coating such as fillers, dyes or pigments, flavors, sweeteners and the like components. Examples of such further ingredients are lactose, titanium dioxide, starch and the like.

Particularly suited as coating materials for the dosage forms of the invention are the Opadry™ materials which mainly contain the before mentioned materials and further ingredients such as plasticizers, e.g. polyethylene glycol.

The dosage forms of the invention have a particular dissolution rate in vitro, said dosage forms providing an effective therapeutic effect for a sufficiently long period of time, in particular for at least 12 hours more in particular for about 24 hours after oral administration.

In particular the oral dosage forms of the invention are suited for dosing every 24 hours.

A further aspect of this invention comprises the finding that the dosage forms, being based on xanthan gum as release controlling agent, allow a strict control of the release of tramadol in function of time. The dosage forms of this invention release tramadol without any peaks or drops in the release pattern of the tramadol active ingredient and the release, moreover, is very reproducible. It has been found that the release of tramadol from the dosage forms of this invention follow a release pattern that is first order or more or less first order. By varying the amount of xanthan gum in a particular dosage form with a given amount of tramadol, the release profile can be influenced. Increasing the amount of xanthan gum will cause a slower release and vice versa. This allows steering the release of tramadol in a controlled manner. For example, it allows dosage forms in which the release of tramadol is complete after a specified time period, e.g. after 6, 12, 18 or 24 hours.

Still a further aspect comprises the finding that for a given amount of xanthan gum, a particular release profile of tramadol is obtained, which remains the same or substantially the same, independently of the amounts of other ingredients in the dosage form, insofar the latter are not ingredients that possess controlled or sustained release properties. This means that for a given amount of tramadol and of xanthan gum, the quantity of the additional ingredients can be changed without the release profile of tramadol being changed or substantially being changed. A substantially the same release profile means that the released quantities of tramadol at specific points in time vary within limits of ±10%, or in particular within limits of about ±5%.

Examples of additional ingredients that can be added without changing the release profile of tramadol are any materials that do not form a controlled release matrix or have some form of interaction with the active ingredient (complexation, addition, etc.)

### EXAMPLES

### Example 1

### Formulation Examples:

| | |
|---|---|
| Active and Excipients | mg/Tablet |

Formulation 1:

| | |
|---|---|
| Tramadol HCl | 90.00 |
| Xanthan Gum | 160.00 |
| Lactose | 94.92 |
| Magnesium Stearate | 3.50 |
| Silicon Dioxide | 1.58 |
| Total | 350.00 |

Formulation 2:

| | |
|---|---|
| Tramadol HCl | 10.00 |
| Xanthan Gum | 120.00 |
| Lactose | 214.93 |
| Magnesium Stearate | 3.50 |
| Silicon Dioxide | 1.57 |
| Total | 350.00 |

Formulation 3:

| | |
|---|---|
| Tramadol HCl | 10.00 |
| Xanthan Gum | 120.00 |
| Lactose | 165.65 |
| Magnesium Stearate | 3.00 |
| Silicon Dioxide | 1.35 |
| Total | 300.00 |

Formulation 4:

| | |
|---|---|
| Tramadol HCl | 66.66 |
| Xanthan Gum | 200.00 |
| Lactose | 28.84 |
| Magnesium Stearate | 3.00 |
| Silicon Dioxide | 1.50 |
| Total | 300.00 |

Formulation 5:

| | |
|---|---|
| Tramadol HCl | 100.00 |
| Xanthan Gum | 300.00 |
| Lactose | 43.25 |
| Magnesium Stearate | 4.50 |
| Silicon Dioxide | 2.25 |
| Total | 450.00 |

Dry blend preparation prior to compression.

After blending the dispensed amount of tramadol HCl, xanthan gum and lactose, a further blending follows after adding of the dispensed and sieved amounts of magnesium stearate and silicon dioxide. The compression of tablets is performed on a rotary press tablet machine.

### Example 2

Dissolution Rate:

The in vitro dissolution rate of Formulation 1 as described in Example 1 was measured according to Ph. Eur. Paddle Method (USP App. 2) at 75 rpm. The dissolution tests were performed on the tablets in 900 ml 0.05 M phosphate buffer with a pH value of 6.8 (USP) at 37° C. A sinker device was used to avoid the sticking of tablets to the vessel or the floating of the tablet. The detection was performed by using high performance liquid chromatography (HPLC) with a refractive index detector for the detection of the active compound. For an in situ measurement of the release rate, a fiber optic dissolution system was used, using the second derivative correction method at the wavelength range of 283 to 289 nm. The dissolution profile can be described as follows:

About 25% Tramadol released after 1 hour,

About 35% Tramadol released after 2 hours,

About 50% Tramadol released after 4 hours,

About 70% Tramadol released after 8 hours,

About 80% Tramadol released after 12 hours,

About 92% Tramadol released after 18 hours

About 100% Tramadol released after 24 hours.

The percentages mentioned above are by weight.

1. A sustained release oral pharmaceutical dosage form which is a pill capsule or tablet containing an effective amount of tramadol, or a pharmaceutically acceptable salt thereof, dispersed in a matrix characterized in that the matrix comprises xanthan gum.

2. A dosage form according to claim 1 wherein tramadol is present in its hydrochloride salt form.

3. (canceled)

4. A dosage form according to claim 1 wherein the matrix contains from about 20% to about 90%, in particular from about 30% to about 80% of xanthan gum.

5. A dosage form according to claim 1 wherein the release of tramadol is controlled by the quantity of xanthan gum, independently of the quantity of other ingredients that do not influence the release of tramadol.

6. A dosage form according to claim 1 wherein the dosage form contains from about 10 mg to 100 mg tramadol hydrochloride, or an equivalent amount of tramadol base or salt form, per tablet

7. A dosage form according to claim 4 which is a tablet and wherein said dosage form is coated with a taste masking coating.

8. A dosage form according to claim 1 for administration on a once-a-day basis.

9. A process for manufacturing an oral dosage form as claimed in claim 1 comprising mixing tramadol hydrochloride in solid form with xanthan gum and optional other ingredients and converting the mixture into the desired dosage form.

10. A dosage form according to claim 1 prepared by direct compression.

11. A process for manufacturing an oral dosage form as claimed in claim 9 comprising mixing tramadol hydrochloride in solid form with xanthan gum and optional other ingredients and converting the mixture into a tablet by direct compression.

12. A dosage form according to claim 2 wherein the matrix contains from about 20% to about 90%, in particular from about 30% to about 80% of xanthan gum.

13. A dosage form according to claim 2 wherein the release of tramadol is controlled by the quantity of xanthan gum, independently of the quantity of other ingredients that do not influence the release of tramadol.

14. A dosage form according to claim 4 wherein the release of tramadol is controlled by the quantity of xanthan gum, independently of the quantity of other ingredients that do not influence the release of tramadol.

15. A dosage form according to claim 12 wherein the release of tramadol is controlled by the quantity of xanthan gum, independently of the quantity of other ingredients that do not influence the release of tramadol.

16. A dosage form according to 2 wherein the dosage form contains from about 10 mg to 100 mg tramadol hydrochloride, or an equivalent amount of tramadol base or salt form, per tablet.

17. A dosage form according to claim 4 wherein the dosage form contains from about 10 mg to 100 mg tramadol hydrochloride, or an equivalent amount of tramadol base or salt form, per tablet.

18. A dosage form according to claim 12 wherein the dosage form contains from about 10 mg to 100 mg tramadol hydrochloride, or an equivalent amount of tramadol base or salt form, per tablet.

19. A dosage form according to claim 13 wherein the dosage form contains from about 10 mg to 100 mg tramadol hydrochloride, or an equivalent amount of tramadol base or salt form, per tablet.

20. A dosage form according to claim 5 wherein said dosage form is coated with a taste masking coating.

21. A dosage form according to claim 12 wherein said dosage form is coated with a taste masking coating.
